(19) United States
(12) Patent Application Publication
(10) Pub. No.: US 2014/0124425 A1
(43) Pub. Date: May 8, 2014

(54) CAP FOR A BUBBLE TRAP

(71) Applicant: Naoufel Bou Akez, El Manar - 1 (TN)

(72) Inventor: Naoufel Bou Akez, El Manar - 1 (TN)

(21) Appl. No.: 14/007,965

(22) PCT Filed: Mar. 22, 2012
(86) PCT No.: PCT/TN2012/000001

§ 371 Date: Jan. 16, 2014

(30) Foreign Application Priority Data
Mar. 31, 2011 (TN) TN2011/0154

(51) Int. Cl. A61M 1/36

(52) U.S. Cl. CPC A61M 1/3627
USPC 210/188

(57) ABSTRACT

The invention relates to an element (12) for a bubble trap (4, 10) of an extracorporeal hemodialysis circuit (1). The element comprises: a body defining a space inside the element (12); an inner wall (14) separating the inner space of the element (12) into first (15) and second (16) compartments; a pressure port (62) which can be connected to a pressure sensor, in order to measure the pressure inside the bubble trap (4, 10); and a discharge opening (63) for discharging clots (C) located in the bubble trap (4, 10). According to the invention, the pressure port (62) opens into the first compartment (15) of the element (12), while the discharge opening (63) opens into the second compartment (16) of the element (12). In addition, the element (12) includes an air intake (66) which is provided in the partition (22) and which opens into the second compartment (16) of the element (12).

### GENERAL TECHNICAL FIELD

This invention relates to the field of extracorporeal hemodialysis devices for use on arterial and venous lines, and more particularly venous bubble trap components or arterial expansion chamber components used in such devices.

In particular, it relates to the respective upper parts of a venous bubble trap or an arterial expansion chamber that can be made in the form of caps or integrated directly in the form of a venous bubble trap chamber or an arterial expansion chamber respectively.

### STATE OF THE ART

Conventional extracorporeal hemodialysis circuits are frequently faced with problems caused by the formation of clots in the extracorporeal hemodialysis circulation. These clots obstruct the different parts making up the circuit or limit the circulating flow, then requiring that the hemodialysis session should be stopped.

In order to overcome these disadvantages, patent application WO 2006/054957 in the name of the applicant discloses an improved extracorporeal hemodialysis circuit, comprising means of extracting blood clots that form in the circuit.

This document discloses an extracorporeal hemodialysis circuit like that shown in FIG. 1.

In this extracorporeal hemodialysis circuit 1, blood is taken from a patient P, enters through an arterial line and is drawn by a pump 3 through an arterial expansion chamber 4, a dialysis machine 5, a venous bubble trap 6, a venous line 7 and a recirculation line 8.

Blood is returned to the patient P through the venous line 7.

The venous bubble trap 6 comprises a supply orifice 61, a pressure port 62, a discharge opening 63 and an outlet conduit 64. A filter 65 is placed at the outlet conduit 64 and is likely to be degraded by blood clots forming in the circuit. The venous bubble trap 6 also comprises an air inlet 66 for air intake and outlet. The outlet conduit 64 is connected to the venous line 7, while the discharge opening 63 is connected to the recirculation line 8 that is also connected to the arterial line 2 upstream of the pump 3.

If there are any clots in the venous bubble trap 6, the user will rinse the circuit with serum and then obstruct the venous line 7 so as to increase the serum level in the venous bubble trap 6 until it reaches the discharge opening 63.

Serum is thus directed to the recirculation line 8 so that the clot can be trapped for example with a clot trap 9 placed in the recirculation line 8.

Once the clot has been evacuated from the venous bubble trap 6, the user no longer closes the venous line 7 and serum is replaced by blood, once again channelled to the patient P to continue the hemodialysis session.

However, this device has disadvantages during its operation.

The rise in the serum level in the bubble trap 6 also causes an increase in the level of serum in the other conduits opening up at the top of the bubble trap, particularly in the pressure port 62 to which a pressure sensor is connected, which can cause significant degradation in the circuit. In particular, it can cause contamination of the hemodialysis generator, or distort the venous pressure read by the pressure sensor.

Furthermore, when blood is being channelled or more generally when a fluid is being channelled in the recirculation line 8, air is also drawn with it and therefore through the various elements of the circuit 1 which is conducive to the formation of new clots.

Patent application WO 2006/054957 in the name of the applicant in 2006 does not solve these problems because it cannot successfully release air from the venous bubble trap without the fluid coming into direct contact with the orifice of the pressure sensor conduit.

### PRESENTATION OF THE INVENTION

This invention aims to disclose a device that does not have these disadvantages.

In particular, this invention is aimed at disclosing a device that can be used to release air in a bubble trap without the fluid (serum or blood) coming into direct contact with the pressure port or with the pressure sensor.

To achieve this, the invention discloses a bubble trap element in an extracorporeal hemodialysis circuit comprising:
a body defining a space internal to said element;
an inner wall dividing the inner space of the element into a first and a second compartment;
a pressure port adapted to be connected to a pressure sensor in order to measure the pressure inside the bubble trap;
a discharge opening to evacuate clots located inside the bubble trap;
   the pressure port opening up in the first compartment of the element,
   the discharge opening opening up in the second compartment of the element characterised in that
   said element also comprises an air intake that opens up into the second compartment of the element.

According to the invention, the bubble trap element may be made by means of a cap fixed to a bubble trap chamber by connection means, or it may be an element integrated into a bubble trap chamber forming the upper part of this chamber.

According to particular embodiments, the bubble trap element has one or several of the following characteristics taken independently or in combination:
the second compartment has a convergent form towards the discharge opening, typically comprising a substantially conical shaped conduit leading to the discharge opening,
the body has a peripheral wall with an approximately circular cross-section above which there is a partition, and the inner wall extends from the partition along a diameter of said peripheral wall;
the conduits extend along the line of said openings and the air intake.

The invention also relates to an arterial or venous bubble trap comprising an element like that described above.

The invention also relates to an extracorporeal hemodialysis circuit fitted with such an arterial or venous bubble trap.

According to one particular embodiment, this extracorporeal hemodialysis circuit comprises:
an arterial line on which there are a pump, an arterial bubble trap and a dialysis machine in this order, and that is connected to a venous bubble trap supply orifice;
a venous line connected to the outlet orifice of the venous bubble trap;
a recirculation line fitted with a clot trap, connected to the discharge opening of the venous bubble trap and the arterial line upstream of the pump, and the arterial line upstream of the pump.

### PRESENTATION OF THE FIGURES

Other characteristics, purposes and advantages of the invention will become clear from the following description that is given for illustrative purposes alone and is in no way limitative, and thus must be read with regard to the appended drawings in which:

FIG. 1 previously presented shows an extracorporeal hemodialysis circuit according to the state of the art,

FIG. 2 shows a bubble trap cap according to one embodiment of the invention,

FIG. 3a shows such a cap associated with a venous bubble trap;

FIG. 3b shows an element of a venous bubble trap in the form of an element integrated into the chamber of a bubble trap;

FIG. 4 shows an extracorporeal hemodialysis circuit with a venous bubble trap according to one aspect of the invention;

FIGS. 5a to 5e show different steps in the use of a venous bubble trap according to one aspect of the invention;

FIGS. 6 and 7 show two views of one embodiment of the venous bubble trap according to one aspect of the invention;

FIG. 8 shows an arterial bubble trap according to one aspect of the invention.

### DETAILED DESCRIPTION

The following description relates to an element of a bubble trap according to one aspect of the invention. This element is preferably a cap like that described below, but the invention also covers a bubble trap element integrated into such a bubble trap.

Furthermore, in the following, the terms “arterial bubble trap” could be understood as an “arterial expansion chamber (AEC)” and vice versa.

FIG. 2 shows a bubble trap cap according to one aspect of the invention.

As shown, the cap 12 comprises a body formed from a peripheral wall 21 with one connecting end 13 provided with connection means and adapted to fit onto a corresponding end of the bubble trap chamber, and one end closed off by a partition 22.

The body of the cap and more particularly the peripheral wall 21 and the partition 22 thus define an inner space inside said cap 12.

In the embodiment shown, the cap 12 comprises a supply orifice 61, a pressure port 62, a discharge opening 63 and an air intake 66.

The cap 12 is also provided with an inner wall 14 dividing the inner space of the cap 12 into two compartments 15 and 16. The inner wall 14 typically extends from the partition 22 and over the entire width of the cap, for example diametrically when the cap 12 has a circular cross-section.

The pressure port 62 opens up in the first compartment and is adapted to be connected to pressure measurement means, typically a manometer.

The discharge opening 63 opens up into the second compartment 16 and is adapted to enable evacuation of the blood clots.

The air intake 66 opens up into the second compartment 16 and is adapted to enable air inlet or outlet from the inner space of the cap 12, and more particularly from the second compartment 16.

The supply orifice 61 opens up into the first compartment 15, and is typically closed off or connected to a blood or serum supply line depending on whether the cap 12 is associated with an arterial expansion chamber or a venous bubble trap.

In the embodiment shown, the discharge opening 63, the air intake 66, the pressure port 62 and the supply orifice 61 are formed in the partition 22.

The two compartments 15 and 16 of the cap 12 are separated from each other by the wall 14 such that fluid or gas exchanges between these two compartments necessarily bypass the wall 14 at is lower end, opposite the openings 61, 62 and 63.

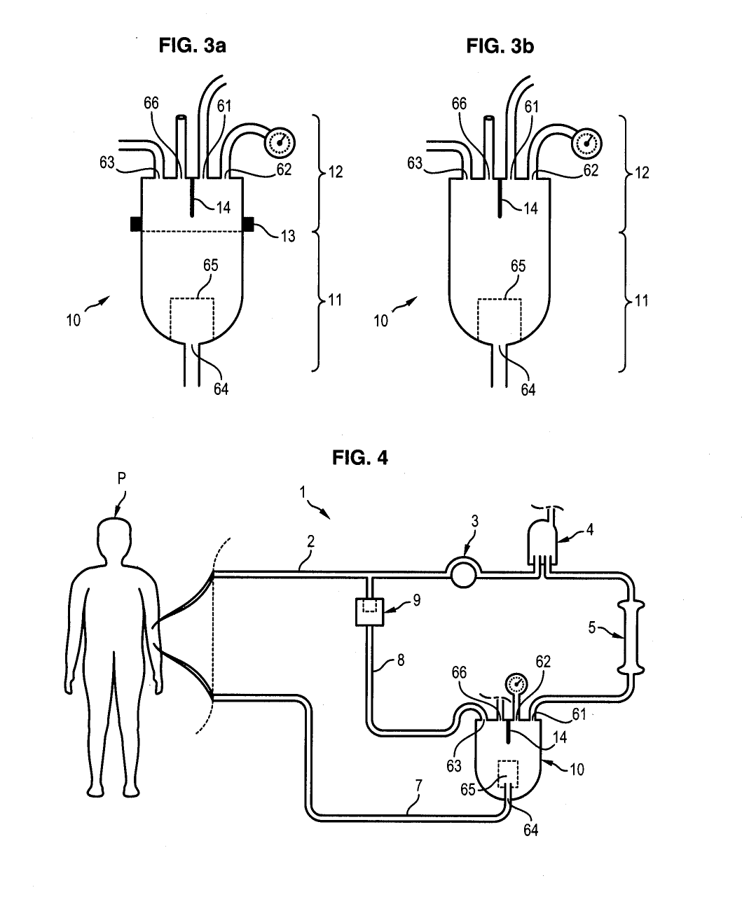

FIG. 3a shows a view of the cap 12 associated with a venous bubble trap chamber.

The venous bubble trap 10 as shown is thus composed of a chamber 11 above which there is a cap 12.

The chamber 11 comprises an outlet conduit 64 carrying blood to the venous line above which there is a filter 65.

As mentioned above, the venous bubble trap 10 may be made in one or several parts; the element 12 possibly but not necessarily being fixed to the chamber 11.

One embodiment in which the element 12 is integrated into the chamber 11 of the venous bubble trap is shown in FIG. 3b, in which the element 12 is shown near the top of the chamber 11 and for example is made in a single piece with the chamber. In this embodiment, the bubble trap element 12 does not in this case include any connection means with the chamber 11.

The venous bubble trap 10 is held in place in a substantially vertical position such that the outlet conduit 64 is at the bottom while the cap 12 is near the top. The blood transported in the venous bubble trap 10 through the supply orifice 61 then drops by gravity towards the outlet orifice 64.

If there is a blood clot in the venous bubble trap 10, the user will rinse the extracorporeal circulation with serum and then will stop the pump, obstruct the outlet conduit 64 of the venous bubble trap 10, open the air intake orifice 66, which makes the serum rise up in the venous bubble trap 10, while air initially present in the venous bubble trap 10 is evacuated through the air intake orifice 66.

Since the two compartments 15 and 16 are separated by the inner wall 14, once serum reaches the level of the inner wall 14, air present in the first compartment 15 cannot enter the second compartment 16 to escape through the air intake 66.

Thus, once the serum level reaches the inner wall 14, it no longer rises in the first compartment 15 because the air present in this compartment can no longer escape from it, whereas air present in the second compartment 16 escapes through the air intake 66 and thus allows the serum level to continue to rise in the second compartment 16 alone.

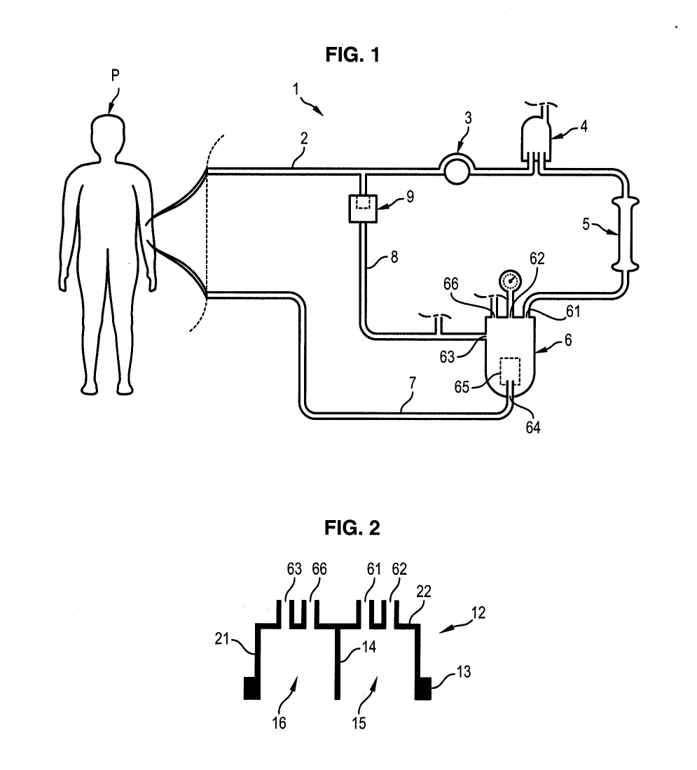

The serum level in the second compartment 16 thus rises until it reaches the discharge opening 63 through which the serum is brought to a recirculation channel 8 similar to that presented previously in FIG. 1, and the clot is brought to a clot collector located on this recirculation channel 8.

FIG. 4 shows an extracorporeal hemodialysis circuit 1 fitted with a venous bubble trap 10 according to one aspect of the invention as presented above.

The circuit 1 shown in this figure comprises elements similar to those shown previously in FIG. 1, particularly:
an arterial line 2,
a pump 3,
an arterial bubble trap or arterial expansion chamber (AEC) 4,
a dialysis machine 5,
a venous bubble trap 10,
a venous line 7,
a recirculation line 8 and a clot trap 9.

Blood is extracted from the patient P through the arterial line 2 and is drawn by the pump 3 into the circuit 1.

Blood thus passes through the arterial expansion chamber (AEC) 4, the dialysis machine 5, the venous bubble trap 10 from which it is directed either into the venous line 7 to be re-injected into the body of the patient P, or into the recirculation line 8 to circulate once again in the circuit 1.

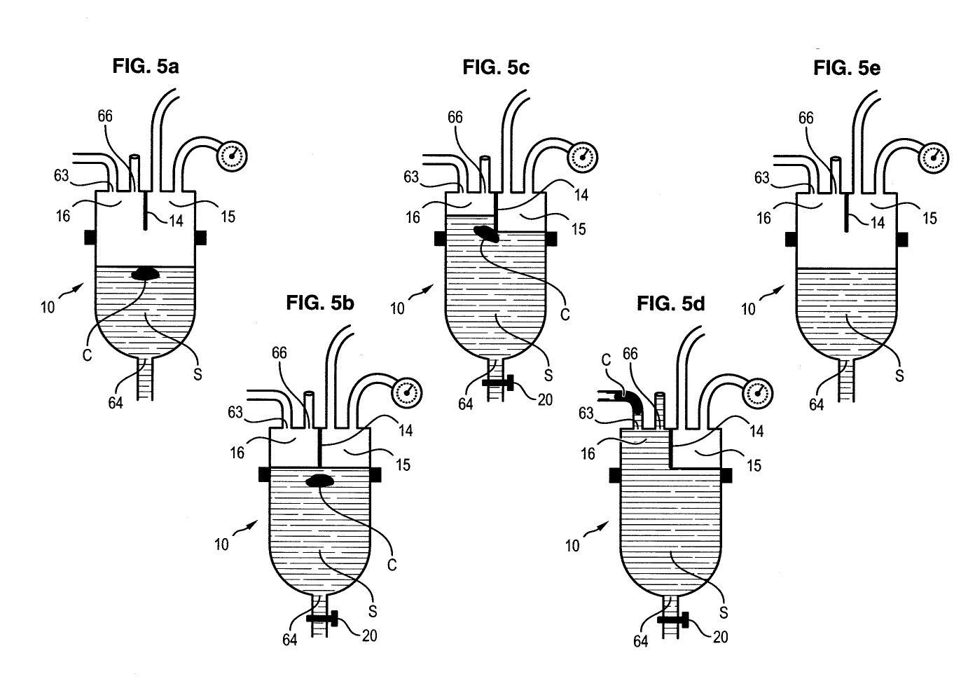

FIG. 5 show several steps in which blood S rises in the venous bubble trap 10 and in which the clot C of blood located in the venous bubble trap 10 is evacuated, according to one aspect of the invention.

FIG. 5a shows the venous bubble trap 10 in which there is a volume of blood S, and a clot C formed in this volume of blood.

In response to the presence of this clot C, the user rinses the extracorporeal circuit with serum, stops the pump, obstructs the outlet conduit 64 of the venous bubble trap, typically using a clamp 20, so as to increase the level of serum S contained in the venous bubble trap 10. Air present in the venous bubble trap in the space that will be filled with serum S is evacuated through the air intake 66.

Once the level of serum S has reached the inner wall 14, it remains at this level in the first compartment 15 and continues to rise only in the second compartment 16; air contained in this second compartment 16 is all that is evacuated through the air intake 66 that opens up in this second compartment 16 as shown in FIGS. 5b and 5c.

The clot C is then evacuated from the venous bubble trap 10 through the discharge opening 63, with serum that is typically transported to the recirculation line 8 so that it can be captured by the clot trap 9. In this way, when the clot C is evacuated from the venous bubble trap 10, there is no longer any air present in the second compartment 16 and therefore no air passes in the recirculation line 8.

The user then stops blockage of the outlet conduit 64 from the venous bubble trap, and the volume of blood S drops while air is reintroduced into the venous bubble trap 10 through the air intake 66.

The venous bubble trap 10 as shown and more precisely the cap 12 thus prevents fluid (serum or blood) from rising in the pressure port 62 and deteriorating the measurement instruments. It also limits the air quantity introduced into the extracorporeal hemodialysis circuit 1 through the recirculation line 8 which thus reduces the formation of clots in the circuit 1.

The steps shown in FIGS. 5a to 5e also apply in the case where the cap 12 is associated with an arterial expansion chamber AEC 4, or in the case where the upper part of such an arterial expansion chamber has the same configuration as the cap 12.

In this particular application, the supply orifice 61 is eliminated or obstructed whereas the blood or serum inlet into the bubble trap as well as their outlet typically passes through the lower part of the arterial expansion chamber AEC 4.

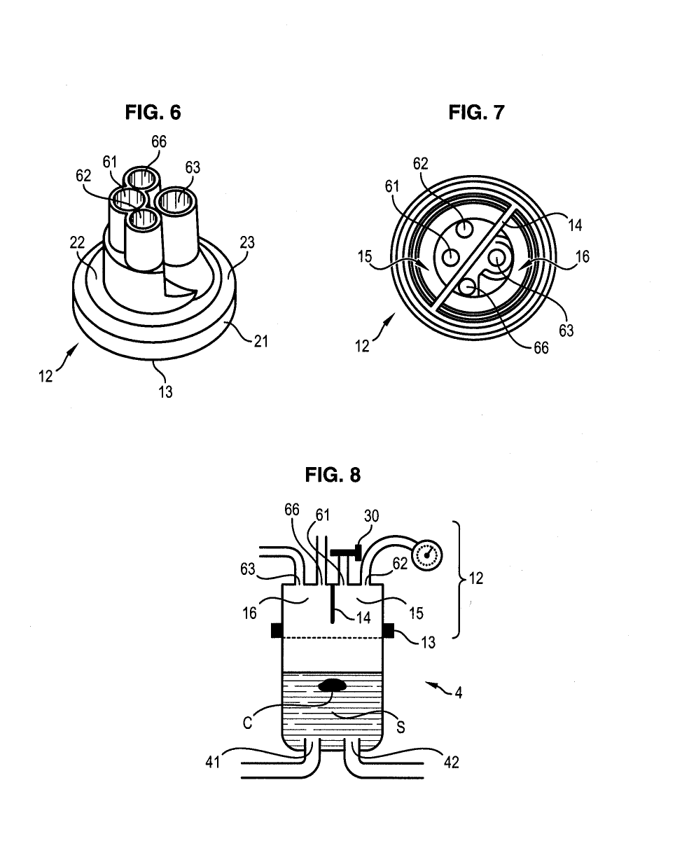

FIGS. 6 and 7 show two views of an embodiment of the cap 12.

FIG. 6 shows a 3D model of the cap 12, whereas figure shows a bottom view of the cap 12 in which the compartments 15 and 16 can be seen.

In the embodiment shown, the peripheral wall 21 is approximately in the shape of a cylinder of revolution and is connected to the partition 22 through a chamfer 23.

The cap 12 comprises conduits extending from the partition 22 in line with the supply orifice 61, the pressure port 62, the discharge opening 63 and the air intake 66.

The conduits are typically substantially in the form of a cylinder of revolution and they have standardised dimensions in order to enable connection of the different lines of the extracorporeal hemodialysis circuit 1 using standard connection means.

The openings 61, 62, 61 and the air intake 66 as shown are substantially in the form of cylinders of revolution, with an axis parallel to the axis of the cap 12.

FIG. 7 shows the distribution of openings 61, 62, 63 and the air intake 66 in the two compartments 15 and 16 formed in the inner space of the cap 12 by the inner wall 14;
the supply orifice 61 and the pressure port 62 open up into the first compartment 15,
the discharge opening 63 and the air intake 66 open up into the second compartment 16.

In the embodiment shown, the second compartment 16 of the cap 12 has an internal geometry converging towards the discharge opening 63 so as to guide the clot C into this conduit 63.

For example, the cap 12 may have a substantially conical shaped conduit leading from the second compartment 16 to the discharge opening 63.

In the embodiment shown, the air intake 66 and the supply orifice are arranged substantially tangent to the inner wall 14.

The discharge opening is at a slight distance from the inner wall 14, to enable formation of the conical conduit described above.

The supply orifice 61, the pressure port 62, the discharge opening 63 and the air intake 66 are typically provided with standard assembly means, so that elements of the hemodialysis circuit can be used with them.

The cap 12 as presented can also be associated with an arterial bubble trap 4 also called an arterial expansion chamber AEC.

FIG. 8 shows an example of adaptation of the cap 12 onto an arterial bubble trap 4.

The arterial bubble chamber AEC 4 comprises an inlet 41 and an outlet 42.

The inlet 41 is connected to the arterial line 2 and it is used to allow blood to enter the arterial bubble trap 4. Blood is then evacuated through the outlet 42 to the dialysis machine 5.

In the embodiment shown, the inlet 41 and the outlet 42 are located in the lower part of the arterial expansion chamber AEC 4, in other words the part opposite the cap once it has been positioned on the arterial expansion chamber 4.

The cap 12 as previously presented may also be used on such an arterial expansion chamber AEC 4 for evacuation of the blood clots located in it.

In the same way as for the venous bubble trap, partition of the cap 12 into two compartments 15 and 16 can evacuate blood clots through the upper part of the bubble trap without damaging pressure measurement means connected to the pressure port 62. In this particular application, the supply orifice 61 of the cap 12 is eliminated.

The cap 12 as presented therefore enables the evacuation of a clot located in an arterial bubble trap

AEC 4 or a venous bubble trap 10 of an extracorporeal hemodialysis circuit 1, by increasing the level of the fluid (blood or serum) in the bubble trap 4 or 10 while protecting pressure measurement instruments that are present due to division of the cap 12 into two internal compartments 15 and 16 and only allowing the fluid level (blood or serum) to rise in one 16 of these internal compartments.

According to one advantageous embodiment, the cap 12 is made from a transparent material so that the user can detect the presence of clots in the bubble trap once the cap 12 is put into position. This clot becomes particularly obvious when the extracorporeal circuit is rinsed with 200 cc of serum, so that it can be eliminated.

The cap 12 typically has a diameter of between 19 and 30 mm depending on the make of the hemodialysis generator used.

The inner wall 14 dividing the inner space of the cap 12 is typically between 5 and 15 mm high. The air intake 66, the pressure port 62 and the supply orifice 61 are typically circular in cross-section and the diameter is between 3 and 6 mm.

The discharge opening 63 typically has a circular cross-section with a diameter larger than the diameter of the other openings 61, 62 and 66, for example between 6 and 8 mm.

The description presented above relates to an extracorporeal hemodialysis circuit 1 in which, blood circulates. It can be easily understood that the cap as presented is not limited to this application and more generally is capable of eliminating a foreign body located in a fluid, typically a serum, circulating in an extracorporeal hemodialysis circuit.

1. Bubble trap (4, 10) element (12) in an extracorporeal hemodialysis circuit (1) comprising
a body defining a space internal to said element (12);
an inner wall (14) dividing the inner space of the element (12) into a first (15) and a second (16) compartment;
a pressure port (62) adapted to be connected to a pressure sensor in order to measure the pressure inside the bubble trap (4, 10);
a discharge opening (63) to evacuate clots (C) located inside the bubble trap (4, 10);
the pressure port (62) opening up in the first compartment (15) of the element (12),
the discharge opening (63) opening up in the second compartment (16) of the element (12), characterised in that
said element (12) also comprises an air intake (66) that opens up into the second compartment (16) of the element (12).

2. Element (12) according to claim 1, wherein the second compartment (16) has a geometry converging towards the discharge opening (63).

3. Element (12) according to the previous claim, wherein the second compartment (16) has an approximately conical shaped conduit leading to the discharge opening (63).

4. Element (12) according to one of the previous claims, wherein the body has a peripheral wall (21) with a substantially circular cross-section over which there is a partition (22), and the inner wall (14) extends from the partition (22) along a diameter of said peripheral wall (21).

5. Element (12) according to the previous claim, wherein the inner wall (14) is between 5 and 15 mm high.

6. Element (12) according to one of the previous claims, including conduits extending along said openings (61, 62, 63) and said air intake (66).

7. Element (12) according to one of the previous claims, characterised in that it is made from a transparent material.

8. Arterial (4) or venous (10) bubble trap comprising an element (12) according to one of the previous claims.

9. Extracorporeal hemodialysis circuit (1) provided with an arterial (4) or venous (10) bubble trap according to the previous claim.

10. Extracorporeal haemodialysis circuit (1) according to the previous claim, comprising:
an arterial line (2) on which there is a pump (3), an arterial bubble trap (4) and a dialysis machine (5) in sequence and which is connected to a supply orifice (61) of the venous bubble trap (10);
a venous line (7) connected to the outlet orifice (64) of the venous bubble trap (10),
a recirculation line (8) provided with a clot trap (9), connected to the discharge opening (63) of the venous bubble trap (10) and to the arterial line (2) upstream of the pump (3).
